(19) United States
(12) Patent Application Publication
(10) Pub. No.: US 2011/0070553 A1
(43) Pub. Date: Mar. 24, 2011

(54) Light Source for a Dental Device

(75) Inventors: Johann Stempfle, Pfaffenhofen (DE); Bernd Gugel, Uim-Einsingen (DE)

(73) Assignee: Kaltenbach & Voigt GmbH, Biberach (DE)

(21) Appl. No.: 12/922,426

(22) PCT Filed: Mar. 13, 2009
(86) PCT No.: PCT/EP2009/001842

§ 371 Date: Dec. 2, 2010

(30) Foreign Application Priority Data
Mar. 14, 2008 (DE) 102008014353.7
Jul. 17, 2008 (DE) 102008033556.8

(51) Int. Cl. A61B 1/24; F21V 15/01; F21V 3/00; F21V 3/02; F21V 3/04; F21V 29/00; A61C 3/00

(52) U.S. Cl. 433/29; 362/362; 362/257; 362/311.06; 362/311.1; 362/311.03; 362/311.04; 362/311.05; 362/373

(57) ABSTRACT

A light source for use in a device, for example in a dental device, comprising a semiconductor element disposed in a housing wherein the semiconductor element comprises a light-emitting surface. The light source further comprises a socket that extends from a side of the semiconductor element opposite the light-emitting surface, the socket being provided for being placed in a frame located in the device, wherein the socket comprises contact elements and lines for power supply to the semiconductor element. The light source is particularly robust and long-lasting.

The invention relates to a light-source for use in an instrument, in particular in a medical or dental instrument. In addition, the invention relates to a medical or dental instrument, in particular a dental turbine handpiece with a light-source of such a type.

In dental instruments a light-source is often provided, in order to illuminate, for example, a machining site, for example a neighborhood of a drill. Customarily halogen illuminating means are employed as light-sources in dental instruments, for example in dental hand-held instruments or turbine handpieces, the light then being guided via a glass rod to a light-exit point of the instrument. These illuminating means are suitable for use in instruments of such a type, inasmuch as they are generally able to generate relatively bright light. One disadvantage, however, is their comparatively limited lifespan. In particular, the halogen lamps are, as a rule, greatly stressed by evolution of heat and by vibrations, this stress subsequently having an effect on their lifespan. The luminous efficiency is also comparatively low. In practice it has become evident that halogen lamps of such a type frequently fail, the treatment has to be interrupted, and a replacement lamp has to be employed.

The object underlying the present invention is to specify a light-source for use in an instrument, in particular in a medical or dental instrument with improved properties, and also a correspondingly improved instrument.

This object is achieved, according to the invention, with the subject-matters stated in the independent claims. Special embodiments of the invention are specified in the dependent claims.

According to the invention, a light-source for use in an instrument, in particular in a medical or dental instrument, is provided which exhibits a semiconductor element arranged in a housing, the semiconductor element exhibiting a light-emission surface; furthermore, the light-source exhibits a base which extends from a side of the semiconductor element situated opposite the light-emission surface and which is provided to be inserted in a socket arranged in the instrument. The base exhibits contact elements and leads for supplying power to the semiconductor element.

A light-source of such a type is particularly suitable for use in a corresponding instrument, in particular in a medical or dental instrument, because in comparison with a halogen illuminating means a semiconductor element is more robust and longer-lasting.

The semiconductor element may be a light-emitting diode (semiconductor chip).

The light-source advantageously further exhibits an optical element for influencing the light radiated from the semiconductor element, the optical element being arranged upstream of the light-emission surface. Furthermore, the optical element advantageously constitutes a light-exit window. The optical element may in this connection be retained on the housing and/or may be integrally connected to the housing.

The optical element is preferably connected to the housing in airtight and/or watertight manner. There may accordingly be provision, for example, that the optical element is hermetically connected to the housing—that is to say, in airtight and watertight manner. By this means, the light-source can be improved as regards its suitability for cleaning; corresponding remarks apply to the instrument equipped with the light-source.

There may be provision that the optical element directly contacts a wall region of the housing over an area.

The optical element is advantageously of plane-parallel, convex, biconvex or spherical shape.

The optical element advantageously consists of glass, sapphire or a light-conducting polymer.

The light-source advantageously further exhibits a support, on which the semiconductor element is arranged, preferably fixed, the contact elements of the base being electrically connected to the support. In this case there may be provision that for this purpose the support exhibits electrical contact areas which are electrically connected to the contact elements.

Advantageously the support is manufactured from an electrically insulating material, preferably from ceramic, silicon or plastic.

Advantageously the support is manufactured from a highly thermally conducting material, preferably from ceramic, silicon or plastic or some other material that conducts heat as well as one of the last-named materials.

The light-source advantageously further exhibits a covering which is arranged on the support and which extends over the semiconductor element. For example, the covering may be a so-called globe top.

The housing further advantageously exhibits an opening, the semiconductor element being arranged in a region of the opening. In this case the opening in the region may exhibit a shape that is matched to the outer shape of the support, preferably at least partly corresponds to this outer shape. For example, the support may exhibit a non-circular shape in horizontal section. By this means, a protection against an incorrect orientation of the support in relation to the housing can be achieved.

The opening may be a through-hole. In this case the region in which the semiconductor element is arranged may be a central region of the through-hole. Alternatively, the opening may be a depression.

According to a further aspect of the invention, a dental instrument is provided that exhibits a light-source according to the invention. Advantageously the base of the light-source is adapted to the socket arranged in the instrument. By this means, a simple exchange of the light-source can be achieved.

According to a yet further aspect of the invention, a light-source is provided for use in an instrument, in particular in a medical or dental turbine handpiece or handpiece and anglepiece. The light-source exhibits a preferably substantially cylindrical housing and also a semiconductor element, arranged in the housing, with a light-emission surface, wherein the housing, consisting of an insulating material, exhibits an opening, facing towards the light-emission surface, which is terminated by a transparent light-exit element.

Several corresponding recesses may also be provided with several corresponding light-sources.

Advantageously in this case the opening is terminated by the light-exit element in airtight and/or watertight manner.

The semiconductor element is advantageously arranged on a support arranged in the housing, in particular on a support made of ceramic. The housing may consist of ceramic.

Furthermore, a dental turbine handpiece or handpiece and anglepiece with an elongated gripping sleeve and also with a turbine head or head drive located at the front end of the gripping sleeve is proposed, the gripping sleeve exhibiting in its circumferential surface a recess in which a light-source according to the invention is arranged. Advantageously the shape of the housing of the light-source and the shape of the recess in the circumferential surface are matched to one another. By this means, a protection against unwanted rotation can be realised.

The invention will be elucidated in more detail in the following on the basis of exemplary embodiments and with reference to the drawings. Shown are:

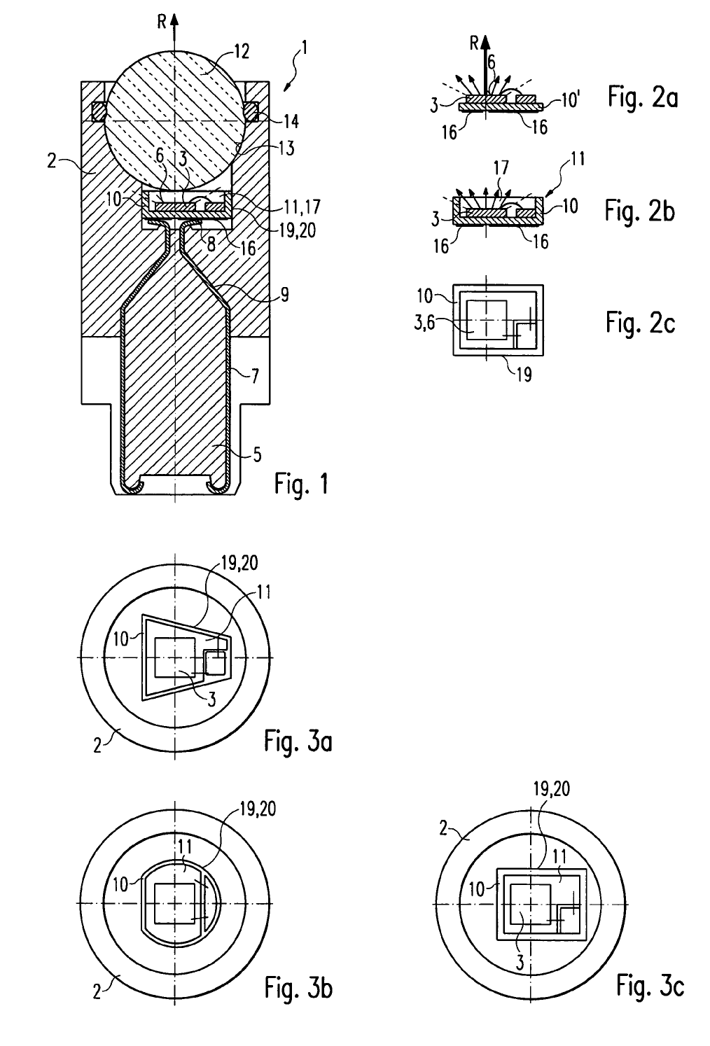

FIG. 1 a cross-sectional sketch of a first exemplary embodiment of a light-source according to the invention,

FIGS. 2a and 2b: sketches with side views relating to two different variants of the support of the semiconductor element,

FIG. 2c a top view of the arrangement shown in FIG. 2b,

FIGS. 3a to 3c: sketches with views from above relating to three different variants of the shape of the support (with the optical element taken away),

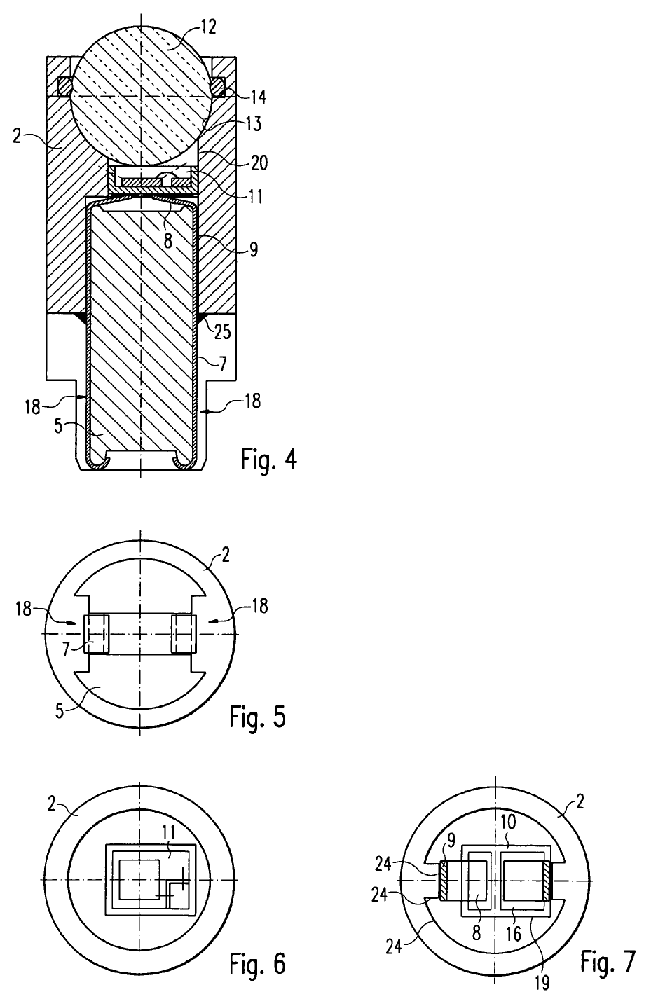

FIG. 4 a cross-sectional sketch of a second exemplary embodiment of a light-source according to the invention,

FIG. 5 a view from below of the light-source shown in FIG. 4,

FIG. 6 a view from above (with the optical element taken away),

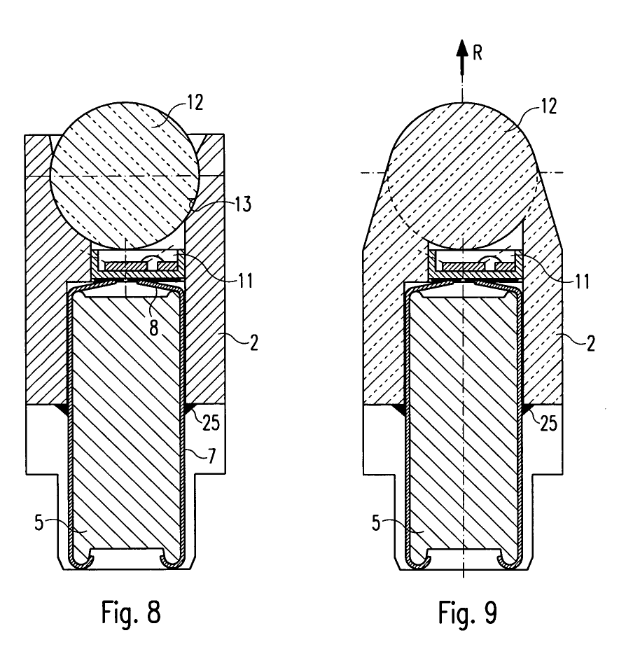

FIG. 7 a view from below (with the base removed),

FIG. 8 a cross-sectional sketch of a third exemplary embodiment of a light-source according to the invention,

FIG. 9 a cross-sectional sketch of a fourth exemplary embodiment of a light-source according to the invention,

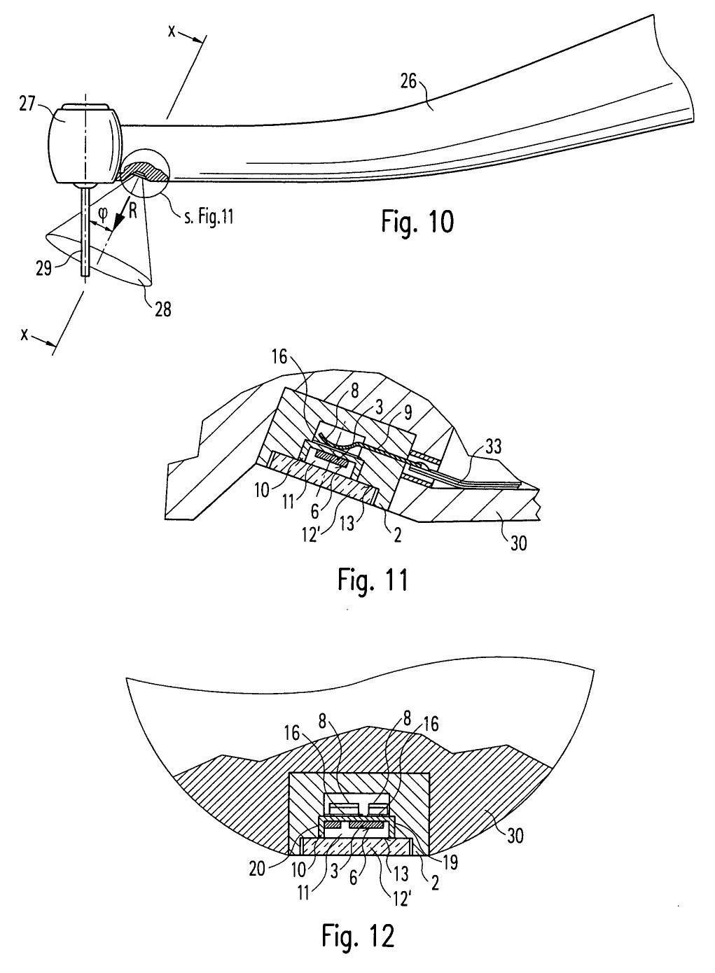

FIG. 10 an exemplary embodiment of a dental turbine handpiece or handpiece and anglepiece with a light-source according to the invention,

FIG. 11 a detail from FIG. 10,

FIG. 12 a sectional representation along the section denoted by x-x in FIG. 10,

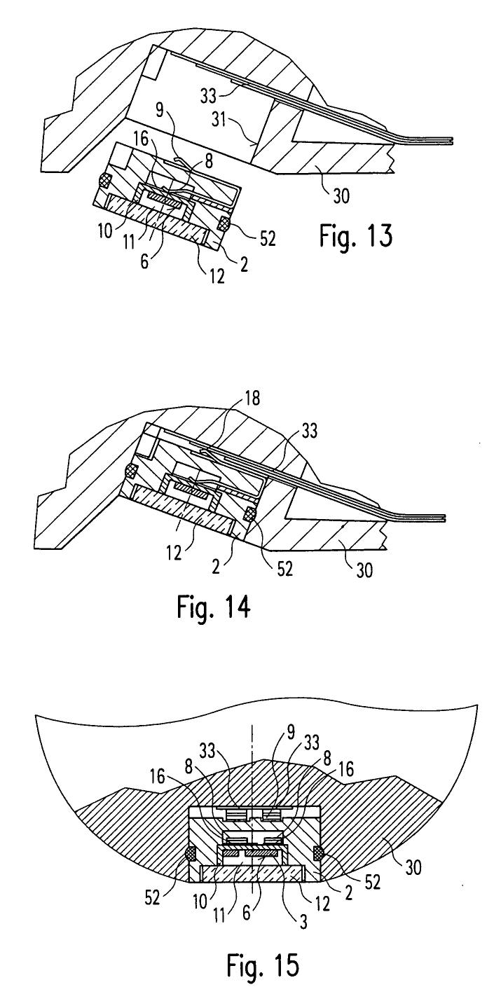

FIG. 13 a sketch relating to a variant of a turbine handpiece with exchangeable light-source, with the light-source taken out,

FIG. 14 as FIG. 13, but with the light-source inserted,

FIG. 15 a sectional representation, corresponding to FIG. 12, of the variant,

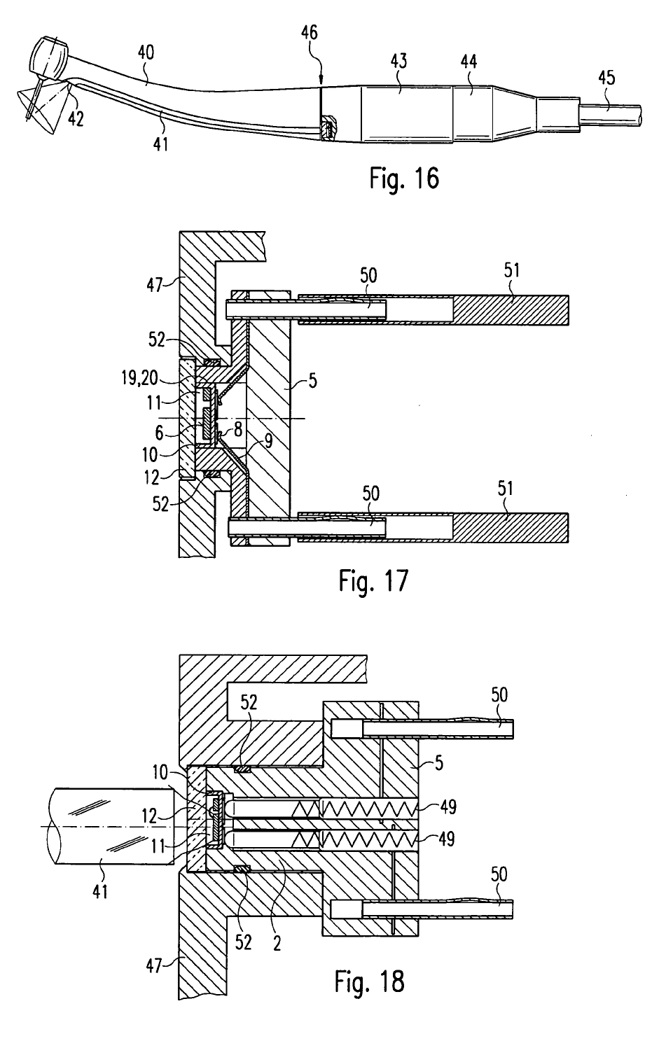

FIG. 16 a dental motor-driven handpiece according to the invention,

FIGS. 17 to 20: sketches relating to different embodiments of the light-source in a motor-driven handpiece,

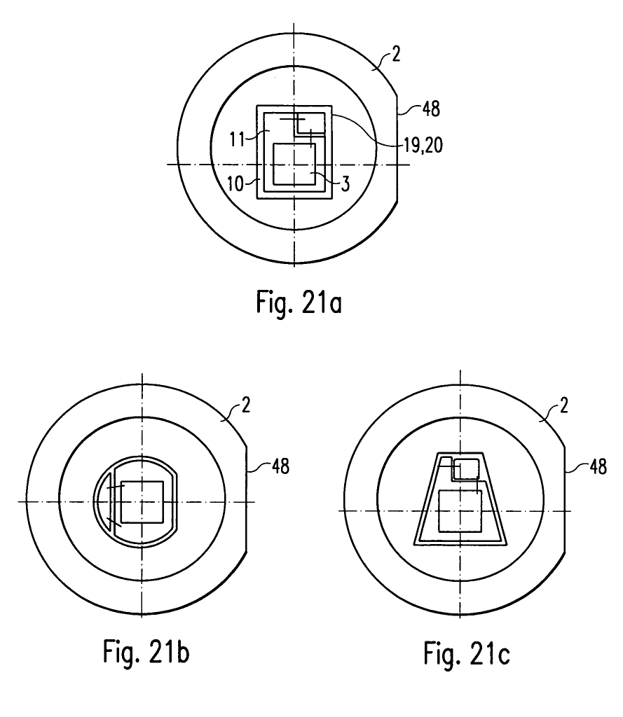

FIGS. 21a to 21c: views, contrary to the principal emission direction, of different variants of the light-source, and

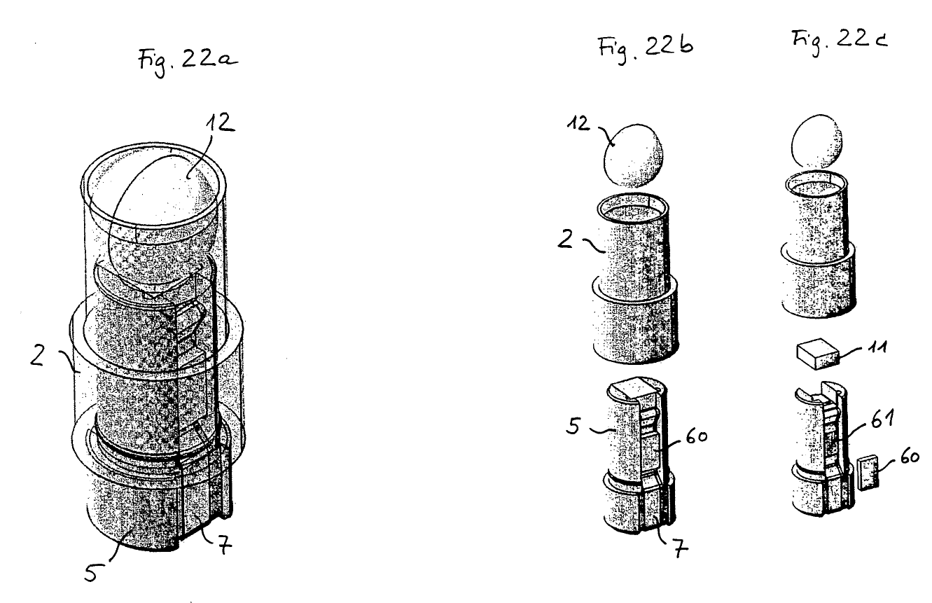

FIGS. 22a to 22c: sketches relating to a fifth exemplary embodiment of a light-source according to the invention.

In FIG. 1, a cross-section through a first exemplary embodiment of a light-source 1 according to the invention is sketched. The light-source 1 is provided for use in an instrument, for example in a medical or dental instrument. But generally the invention is not restricted to use in medical instruments. It may also generally find application, for example, in tools, for example electrical drilling tools.

The light-source 1 includes a semiconductor element 3 which acts as illuminating means—that is to say, a semiconductor element 3 that is able to radiate light, for example a light-emitting diode or a semiconductor chip or light-emitting diode. The semiconductor element 3 exhibits an active surface or, to be more precise, a light-emission surface 6, which may be a flat surface. Perpendicular or normal to the light-emission surface 6 there extends a ‘principal emission direction’ of the light-source, which is indicated in FIG. 1 by an arrow R. For the purpose of easier description, in the following—corresponding to the representation from FIG. 1—the direction of the principal emission direction R is designated by ‘up.’

The semiconductor element 3 is arranged in a housing 2 which is preferably manufactured from an electrically insulating material, for example from plastic, glass or ceramic. The housing 2 preferably consists of a highly thermally conductive material, in order to be able to transport away effectively the heat generated by the semiconductor element 3. The housing 2 may have a cylindrical outer shape.

In the first exemplary embodiment the housing 2 exhibits an opening in the form of a depression, in which the semiconductor element is 3 arranged. In a horizontal section or in a section perpendicular to the principal emission direction the element is consequently surrounded by the housing 2 on all sides.

Furthermore, the light-source 1 includes a base 5 which extends from a side of the semiconductor element 3 located opposite the light-emission surface 6. With reference to FIG. 1, the base 5 consequently extends ‘downwards’ from the semiconductor element 3. The base 5 is provided to be inserted in a socket (not shown in FIG. 1) arranged in the, for example, dental instrument. Advantageously the base 5 may therefore be shaped in such a way that it fits into a corresponding socket such as is provided in existing dental instruments for receiving a known light-source, for example a halogen light-source. This enables a particularly simple exchange of a corresponding known light-source for a light-source according to the invention. In particular, the stated socket may be a plug-in socket.

There may be provision that the dental instrument exhibits a light guide, for example in the form of a glass rod, which is provided for guiding light to a machining site designated for treatment with the instrument, and in this case the light-source according to the invention is arranged in such a manner relative to the light guide that light from the light-source is coupled into the light guide.

The base 5 further exhibits contact elements 8 and electrical leads 7, 9 for supplying power to the semiconductor element 3. The contact elements 8 are constituted by the upper end regions of the electrical leads 9; denoted by reference symbol 7 are those portions of the electrical leads which extend beneath the lower edge of the housing 2; the portions above the portions 7 are denoted by 9.

In the example that is shown, the contact elements 8 with their end regions are arranged between the semiconductor element 3 and the bottom or floor of the depression of the housing 2. In this case they protrude—as indicated in FIG. 1—obliquely upwards and are designed in such a manner that they can be resiliently bent downwards—that is to say, in the direction of the floor of the depression.

As indicated in FIG. 1, in the first exemplary embodiment the housing 2 and the base 5 are integrally formed and the electrical leads 7, 9 are accordingly extrusion-coated or potted.

The base 5 may, in particular, be arranged in such a way that with a first partial region, which points towards the semiconductor element 3, it is surrounded by the housing 2, and with a second partial region, which is located opposite the first partial region, it protrudes from the housing 2.

The light-source is suitable to be employed in a corresponding dental instrument. Advantageous in this connection is, inter alia, the fact that the lifespan of a light-emitting semiconductor element is generally distinctly longer than the lifespan of a halogen illuminating means. Also, a semiconductor element generally has a higher efficiency than a halogen lamp. Furthermore, a semiconductor element can generally be constructed to be smaller in comparison with a halogen lamp, so that advantages arise also as regards physical size.

Furthermore, the light-source according to the exemplary embodiment shown here exhibits an optical element 12 for influencing the light emitted by the semiconductor element 3. The optical element 12 in this case is arranged in front of or above the light-emission surface 6 of the semiconductor element 3 and may constitute a light-exit window—that is to say, a light-exit element—so that the semiconductor element 3 is protected by this means. In the example that is shown, the depression of the housing 2 is sealed in the upward direction by the optical element 12. By this means, a protective function is formed for components that are located in the depression below the optical element 3—that is to say, for example, for the semiconductor element 3 and the contact elements 8.

In the first exemplary embodiment shown here, the optical element 12 is retained on the housing 2. For example, as indicated in FIG. 1, a ring element 14—for example in the form of a sealing element made of an elastic material, that is to say, for example, an O-ring—may serve for this purpose.

In addition there may be provision that—as likewise indicated in FIG. 1—the shape of the optical element 12 and the shape of the housing 2 are matched to one another in such a way that a large-area contact between the two components 12, 2 is established when these are located in the designated reciprocal installation positions. In this connection a contact surface 13 can be provided which, for example, defines a partial region of a spherical surface. The optical element 12 in this case accordingly directly contacts the contact surface 13 of the housing 2 provided for this purpose with a partial region of its surface. By this means, a particularly good protective function for the semiconductor element 3 may be formed, such as can be advantageous, for example, in the case of a sterilisation of the instrument.

The optical element 12 and the housing 2 are preferably connected to one another in airtight and/or watertight manner—that is to say, for example, they are hermetically sealed.

Advantageously in this case the optical element 12 can be shaped in a rotationally symmetrical manner and can be arranged in such a way that the axis of symmetry is parallel to the normal to the surface of the light-emission surface 6 of the semiconductor element 3 and preferably runs through a midpoint of the light-emission surface 6.

The optical element 12 may be a lens. The optical element 12 may, for example, be plane-parallel, convex, biconvex or—as indicated in FIG. 1—spherical. The optical element 12 may consist of glass, sapphire or a light-conducting polymer.

In the exemplary embodiment shown, furthermore a support 10 is provided, on which the semiconductor element 3 is arranged and preferably fixed and to which it is electrically connected. The support 10 in this case is electrically connected to the contact elements 8 of the base 5. For this purpose the support 10 may, for example, as is evident in the partial representation from FIG. 2b, exhibit on its underside corresponding contact surfaces 16 which are provided for the purpose of electrical contact with the contact elements 8 of the base 5. The contact surfaces 16 are preferably arranged on the outside of the support 10, specifically on the outer surface located opposite the semiconductor element 3. The contact surfaces 16 may, for example, be applied by means of PVD coating (PVD: physical vapor deposition) or screen printing etc.

The support 10 is preferably manufactured from an electrically insulating material, for example from ceramics, silicon or plastics. A material of such a type is also advantageous to the extent that it conducts heat comparatively well and can consequently contribute towards heat that is generated in operation of the semiconductor element 3 being transported away particularly effectively.

The support may—as indicated in FIG. 2a—have the shape of a plate—that is to say, it may be a pure flat material or, as indicated in FIG. 2b, may have the shape of a pot or ‘bowl’. For the purpose of distinguishing, the variant of the support shown in FIG. 2a is provided with reference symbol 10′.

Particularly in the case of a pot-shaped support 10, for the purpose of protecting the semiconductor element 3 there may be provision that the latter is provided with a covering 17 (globe top) which is arranged on the support 10 or in the interior space formed by the pot and which extends over the semiconductor element 3. The semiconductor element 3 is preferably completely covered by the covering 17. The covering 17 may be constituted by a potting compound. By virtue of the covering 17 the semiconductor element 3 can, in particular, be protected against being touched and against environmental influences such as moisture and the like. Also, any internal wiring of the semiconductor element 3 which is possibly present running within the ‘pot’ can be covered and protected by the covering 17. With respect to its ‘upper’ (according to FIG. 2a) boundary, the covering 17 can be designed in such a manner that it terminates with the lateral edge of the ‘pot’, so that the upper edge of the pot and the upper edge of the covering 17 lie in one plane.

In the following, the unit consisting of the semiconductor element 3, the support 10 and, where appropriate, the covering 17 will also be designated as the ‘illuminating element’ 11.

FIG. 2c shows a top view of the support 10 shown in FIG. 2b in side view. It will be discerned that in this form the support 10 exhibits a rectangular shape or contour in horizontal section. In this case the support 10, together with the covering 17, is accordingly parallelipipedal.

As indicated in FIG. 1, there may be provision that the housing 2 constitutes, for example in the region of the depression, a support socket for the support 10. In this case the shape of the support socket may advantageously be adapted to the outer shape of the support 10. Accordingly, there may advantageously be provision that the shape of the support 10, on the one hand, and the shape of the depression of the housing 2 or of the support socket formed by the housing 2, on the other hand, are matched to one another in such a way that the support 10 with the semiconductor element 3 arranged thereon can be positively inserted into the support socket. In the case shown in FIG. 1, in this sense the depression exhibits in its lower region a wall with rectangular cross-section, which corresponds in shape and size to the outer shape of the support 10. In this manner the support 10 can be arranged in the depression of the housing 2 particularly easily in a defined orientation and position. The contour of the support 10 or of the illuminating element 11 is denoted by reference symbol 19; the contour of the support socket or of the depression is denoted by reference symbol 20.

In particularly advantageous manner there may be provision that the outer shape or horizontal cross-section of the support 10 forms a rectangle, a polygon, a segment of a circle, a circle with groove or cam or such like, as sketched in exemplary manner in FIGS. 3a, 3b and 3c, which each show a view from above with optical element 12 taken away. As FIGS. 3a and 3b show in exemplary manner, by suitable choice of this shape it can, in particular, be ensured that the support 10 can be introduced into the depression only in an unambiguous orientation. In this manner an incorrect electrical contacting between the support 10 and the contact elements 8 of the base 5 can be reliably prevented.

As further becomes clear from FIGS. 3a, 3b and 3c in conjunction with FIG. 1, in the example that is shown the semiconductor element 3 is square in horizontal section, and accordingly the light-emission surface 6 is square, the support 10 being arranged relative to the housing 2 in such a way that the midpoint of the light-emission surface 6 coincides with the emission direction R.

There may furthermore be provision that the support socket constituted by the housing is designed in such a manner that the support 10, guided therein, can be pressed from above against the resilient contact elements 8 of the base 5. In this case there may be provision that upon insertion of the optical element 12 the support 10 is pressed by it with its contact surfaces 16 against the electrical contact elements 8 of the base 5. Furthermore, in this case the depression may be shaped in such a way that a press-in depth of the optical element 12 is limited by the depression or by the support socket.

In FIG. 4, a cross-section through a second exemplary embodiment of a light-source according to the invention is shown. In the following, only differences from the first exemplary embodiment will be dealt with. Unless stated otherwise, the remarks relating to the first exemplary embodiment apply analogously. The same reference symbols have been chosen for like or corresponding components. Corresponding remarks also apply to the further (exemplary) embodiments.

In contrast to the first exemplary embodiment, the opening in the housing 2 is not a depression but a through-hole. In the example that is shown, the through-hole extends from the top downwards along the principal emission direction R. The base 5 and the housing 2 are of bipartite design. For the purpose of retaining the base 5 in relation to the housing 2 an adhesive bond can be provided. In FIG. 2 an appropriate glued joint 25 is indicated.

In the example that is shown, the base 5 with a first partial region is arranged within the through-hole of the housing 2 and with a second partial region protrudes downwards from the housing 2. Denoted by reference symbol 18 is a contact region of the base 5 for connection to the instrument-side drive.

The connection between base 5 and housing 2 is preferably such that the base 5 can only be inserted into the housing in the correct rotary position. For this purpose a corresponding shaping, non-circular in horizontal cross-section, of the base 5 or of the housing 2 may, for example, be provided. An example of this is indicated in FIG. 5, in which a view from below of the base 5 and the housing 2 is shown.

In the second exemplary embodiment the optical element 12 is arranged in such a way that it seals the through-hole on that side of the through-hole which points to the light-emission direction R. For the purpose of sealing and retaining the optical element 12 on the housing 2, once again a ring element 14 made of elastic material, for example an O-ring, can be provided.

FIG. 6 shows a view from above, with the optical element 12 removed, and FIG. 7 shows a view from below, with the base 5 removed. Denoted by reference symbol 24 is the corresponding inner contour of the housing 2 or of the through-hole for receiving the base 5 and for alignment with respect to the illuminating element 11.

The support 10 may be retained in a manner analogous to the first exemplary embodiment. But a projection may also be provided in the through-hole, which serves as a rest or abutment for retaining the support 10.

In FIG. 8 a third exemplary embodiment is sketched. It differs from the second exemplary embodiment in that the optical element 12 is held to the housing 2 by a clamp joint, for example a snap-in joint.

In FIG. 9 a fourth exemplary embodiment is sketched. The special feature in this exemplary embodiment consists in the fact that the optical element 12 and the housing 2 are constructed in one piece. The housing 2 and the optical element 12 may in this case be manufactured from, for example, glass or transparent polymer. The housing 2 in this case exhibits, in a region that points to the principal emission direction R, a conical region which passes over in positive manner into the spherical shape of the optical element 12 chosen here. In this case there may be provision that the optical element 12 is merged with the housing 2 and in this manner forms a unit. An integral connection between the housing 2 and the optical element 12 may accordingly be provided. This embodiment enables a particularly reliable protection of the semiconductor element 3 from the top or from the side of the optical element 12.

In FIGS. 22a to 22c a fifth exemplary embodiment of a light-source according to the invention is represented in sketched manner. FIG. 22a shows a partly transparent representation of the assembled light-source, and FIG. 22b shows the parts constituted by base 5, housing 2 and optical element 12 of the light-source in the separated state. FIG. 22c shows a further representation, in which—in comparison with FIG. 22b-two further components, namely the illuminating element 11 and a series resistor 60, are represented, separate from the base 5.

The contact elements 7, just like the contact elements 8, 9 (not denoted separately in FIGS. 22a to 22c), can be formed on the circumferential surface of the base 5 as a conductor-track structure or, in short, conductor structure. In the lower region of the base 5 they may represent the contacting with respect to the device-side socket.

The optical element 12—for example in the form of a glass sphere—and the base 5 can be connected to the housing 2 via snap-in joints. Additionally, a seal with an adhesive can be provided in each instance.

At particularly high light intensity and/or at particularly high operating temperatures—that is to say, in ‘critical applications’—it is advantageous to provide a series resistor 60 for the operation of the semiconductor element 3, in order to be able to limit the current for the semiconductor element 3—that is to say, the ‘LED current’—and consequently to lessen the risk of a destruction of the semiconductor element or of the illuminating element 11. (Alternatively, a drive with an adjustable voltage-source can be provided for this purpose.) This series resistor 60 is advantageously arranged within the light-source 1 in this manner the light-source 1 can be used particularly easily in existing instruments or lamps. As a result, the compatibility of the light-source is accordingly particularly good. For example, for this purpose—as sketched in FIG. 22c—the base 5 may exhibit a recess 61 in which the series resistor 60 may be arranged. For example, the contact element 7 may be guided by this recess 61, and the series resistor 60 can be electrically connected there to the contact element 7. Both the illuminating element 11 and the series resistor 60 can be connected in an electrically conducting manner to the conductor structure or to the contact elements 7, 8, 9 via soldered connections.

For the purpose of contacting the two components constituted by illuminating element 11 and series resistor 60, resilient contacts can also be provided. By reason of the filigree parts, however, it is advantageous to deposit the contacts and leads directly on the base 5 chemically/galvanically. For this purpose, a plastic that is capable of being activated by means of laser beam can be used for the base, and the conductor structure can be generated (exposed) by laser beam. On the activated surfaces the conductor structure can then be chemically deposited and galvanically thickened up to the requisite thickness (for example, 30 μm Cu/5 μm Ni/1 μm Au).

In FIGS. 10 to 12 a further exemplary embodiment of a light-source according to the invention is shown which is particularly suitable for use in a dental turbine handpiece 26 or handpiece and anglepiece. FIG. 10 shows a schematic view of such a turbine handpiece 26 or handpiece and anglepiece; at the front end of the instrument there can be discerned a turbine head 27 or head drive and a machining tool 29 inserted in the turbine head 27 or head drive, which may be, for example, a drill. FIG. 11 shows a detail view from FIG. 10, specifically from the region marked appropriately in FIG. 10; FIG. 12 shows a sectional representation, specifically along the section denoted by x-x in FIG. 11. Once again, in the following the reference symbols used further above have been adopted, with analogous significance. Unless stated otherwise, the above remarks apply correspondingly.

The light-source exhibits a preferably substantially cylindrical housing 2 and also a semiconductor element 3, arranged in the housing 2, with a light-emission surface 6. The semiconductor element 3 may once again be retained by a preferably ceramic support 10. The housing 2 consists of an insulating material, for example ceramic, and exhibits an opening, facing towards the light-emission surface 6 of the semiconductor element 3, which is terminated by the optical element 12 which in this case may constitute a transparent light-exit element.

The housing 2 is preferably of shallow form. For example, the housing may be cylindrical, in which case the height of the corresponding cylinder is smaller than its diameter.

In this example the light-source is arranged on the instrument—that is to say, here on the dental turbine handpiece 26 or handpiece and anglepiece—in such a way that the optical element 12 directly forms an outer-wall region of the instrument. For this purpose, in the instrument, specifically in a wall 30 of the sleeve of the instrument, in the vicinity of the turbine head 27 or head drive an indentation—indicated in FIGS. 11 and 12 in sketched manner—is provided, in which the light-source is arranged.

The light-source is arranged relative to the remaining turbine handpiece 26 or handpiece and anglepiece in such a manner that it can serve for illuminating a machining site to be machined with the turbine handpiece 26 or handpiece and anglepiece. In FIG. 10 the illuminated region is denoted by reference symbol 28. The illuminated region 28 extends around the principal emission direction R and includes a region around the end of the machining tool 29. The light-source may, for example, be arranged so close to the turbine head 27 that the principal emission direction R forms with the longitudinal axis or axis of rotation of the machining tool 29 an angle φ of maximally 60°, preferably maximally 45°.

The electrical contacting of the light-source can be provided at the bottom of the indentation, for example by means of soldered connection. In FIG. 11 a contacting with the instrument-side drive is denoted by reference symbol 33. This contacting 33 can be provided by means of flexible conductors based on Kapton (covering sheet of support made of Kapton) or by means of MID (MID: moulded interconnect devices), for example on the outer sleeve of the instrument. But the contacting 33 may also be provided inside the turbine handpiece, for example within or in the wall 30.

In this case the optical element 12 is advantageously sealed to the housing 2 with a sterilisable adhesive. The optical element 12 may in this case be constituted by a plane-parallel disc. The optical element 12 may in this case exhibit a prismatic ground surface. The optical element 12 may, for example, consist of glass, sapphire, pressed glass or transparent polymer. The optical axis of the optical element 12 may advantageously be aligned with the designated machining site of the turbine handpiece 26 or handpiece and anglepiece, as indicated in FIG. 10; it may coincide with the principal emission direction R.

In FIGS. 13 to 15 a variant with respect to the last-mentioned light-source is shown. The difference consists, in particular, in the fact that the light-source according to this variant is provided in the turbine handpiece 26 or handpiece and anglepiece in exchangeable manner. By this means, a particularly easy replacement is possible in the case of a defect. In FIG. 13 the situation with the light-source taken out is shown; in FIG. 14 the installed situation is shown. FIG. 15 shows a section at right angles to the longitudinal axis of the turbine handpiece 26 or handpiece and anglepiece.

In the wall 30 of the turbine handpiece 26 or handpiece and anglepiece, which constitutes the circumferential surface of the instrument, for this an indentation or recess 31 is provided which is suitable for receiving the light-source. Preferably the shape of the housing 2 of the light-source and the shape of the recess 31 in the circumferential surface of the instrument are matched to one another. For example, the recess 31 may exhibit a shape corresponding to the outer shape of the housing 2 in such a manner that the light-source can be inserted into the recess 31 only in the designated orientation.

Advantageously an impervious, for example airtight and/or watertight, connection is provided between the light-source and the wall 30. A sealing element 52, for example in the form of an O-ring, may serve for this purpose. This is advantageous, for example, with respect to sterilisability of the turbine handpiece 26 or handpiece and anglepiece.

For the electrical connection between the light-source and the remaining turbine handpiece 26, in this case the resilient contact elements 8 can be provided on the housing 2. For example, the contact region 18 of the contact elements 8 of the light-source can be provided at the bottom of the recess 31.

In FIG. 16 a motor-driven handpiece with a light-source according to the invention is sketched. The motor-driven handpiece includes a handpiece and anglepiece 40, a glass rod 41, which serves as light guide, a light-exit region 42 at the front end of the glass rod 41, a motor part 43, a coupling part 44, a hose part 45. The interface between the handpiece and anglepiece 40 and the motor part 43 is denoted by reference symbol 46. The light-source is preferably provided at the boundary between the handpiece and anglepiece 40 and the motor part 43. The light-source is preferably provided in the motor-driven handpiece in an exchangeable manner.

Sketches relating to different embodiments of the light-source and of the retaining device and electrical connection of the light-source in the motor part are shown in FIGS. 17 to 20.

In the variant shown in FIG. 17, pin bushings 50 for electrical connection of the light-source to the electrical lead 51 inside the motor are provided; the electrical lead 51 inside the motor leads, on the other hand, to the coupling part 44 or to the hose part 45. In this example the pin bushings 50 are connected both to the housing 2 and to the base 5. For the electrical connections to the leads on the motor side, riveted joints, press joints or crimped joints, for example, can be provided.

A sealing element, for example in the form of an O-ring 52, may serve for sealing and fixing the housing 2 in the motor part 43.

In the variant shown in FIG. 18, resilient, preferably cylindrical contact pins 49 are provided for the electrical connection between the illuminating element 11 and the pin bushings 50.

Furthermore, in FIG. 18 the end of the light guide or glass rod 41 facing towards the motor part can be discerned. The relative arrangement between the light-emission surface 6 and the light guide 41 is such that the centre of the light-emission surface 6 lies on the longitudinal axis of the end region of the light guide 41; in this manner, light is coupled particularly effectively from the light-emission surface 6 into the light guide 41.

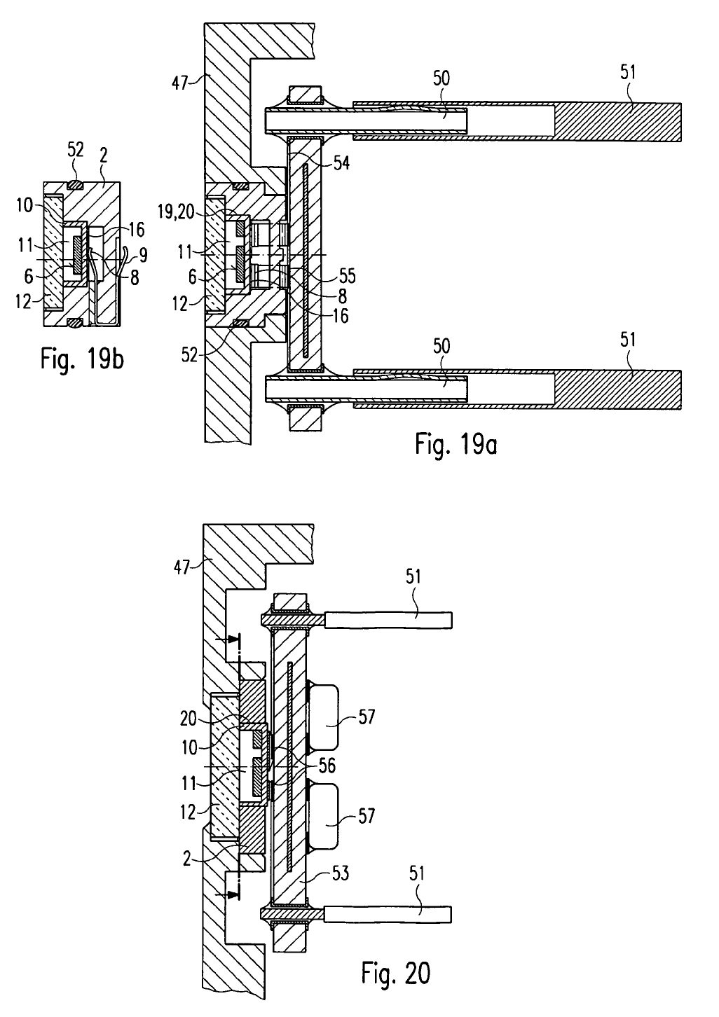

In FIG. 19a a variant with a printed circuit board 53 is shown. The printed circuit board 53 may serve for adapting the supply voltage to the requirements of the semiconductor element 3. The printed circuit board 53 preferably consists of ceramic and exhibits electrical conductors, contact regions and soldering regions. In this case a spring-contact region 55 serves for the electrical connection between the printed circuit board 53 and the illuminating element 11. In FIG. 19b a section the light-source is represented, which in comparison with the representation from FIG. 19a has been rotated by 90°.

In the variant sketched in FIG. 20, soldered joints 20 are provided between the light-source and the printed circuit board 53. The support 10 here is accordingly directly soldered onto the printed circuit board (ceramic substrate). On the side of the printed circuit board 53 located opposite the soldered joints 20 electrical components 57 are provided which form an electrical wiring connection on the printed circuit board 53 for the purpose of level-matching of the semiconductor element 3.

In FIG. 21a a view of the light-source contrary to the principal emission direction is shown. It will be discerned that in this section the housing 2 may exhibit a non-circular contour; for example, the contour may be given by a circular line with a flattened portion 48. The motor-part housing 47 may exhibit a corresponding shape, so that an unambiguous positional orientation between these two components 2, 47 is guaranteed. By this means, a protection against unwanted rotation can be realised which can, in particular, contribute to ensuring the designated electrical contacting. FIGS. 21b and 21c show two further variants. Apart from the flattened portion 48, FIGS. 21a to 21c correspond to FIGS. 3a to 3c.

1. Light-source for use in an instrument, in particular in a medical or dental instrument, exhibiting
at least one semiconductor element arranged in a housing, the semiconductor element comprising a light-emission surface, and
a base that extends from a side of the semiconductor element located opposite the light-emission surface and is provided to be inserted in a socket arranged in the instrument, the base comprising contact elements and leads for supplying power to the semiconductor element.

2. Light-source as claimed in claim 1, further comprising an optical element for influencing the light radiated from the semiconductor element and arranged upstream of the light-emission surface.

3. Light-source as claimed in claim 2, wherein the optical element is retained on the housing and/or is integrally connected to the housing.

4. Light-source as claimed in claim 2, wherein the optical element is connected to the housing in an airtight and/or watertight manner.

5. Light-source as claimed in claim 2, wherein the optical element is of plane-parallel, convex, biconvex, or spherical shape.

6. Light-source as claimed in claim 2, wherein the optical element comprises glass, sapphire, or a light-conducting polymer.

7. Light-source as claimed in claim 1, further comprising a support, on which the semiconductor element is arranged, the contact elements of the base being electrically connected to the support.

8. Light-source as claimed in claim 7, wherein the support is manufactured from an electrical insulating material.

9. Light-source as claimed in claim 7, wherein the support is manufactured from a highly thermally conducting material.

10. Light-source as claimed in claim 7, with a covering arranged on the support and extending over the semiconductor element.

11. Light-source as claimed in claim 1, wherein the housing comprises an opening and the semiconductor element is arranged in a region of the opening.

12. Light-source comprising a support on which the semiconductor element is arranged, the contact elements of the base being electrically connected to the support wherein the housing comprises an opening and the semiconductor element is arranged in a region of the opening, and wherein the opening in the region comprises a shape that is matched to the outer shape of the support.

13. Dental instrument, comprising a light-source as claimed in claim 1.

14. Dental instrument as claimed in claim 13, wherein the base of the light-source is adapted to the socket of the instrument arranged in the instrument.

15. Light-source for use in an instrument, in particular in a medical or dental turbine handpiece or handpiece and anglepiece, exhibiting comprising a preferably substantially cylindrical housing and a semiconductor element arranged in the housing with a light-emission surface, wherein the housing comprises an insulating material and comprises an opening facing towards the light-emission surface (6), which is terminated by a transparent light-exit element.

16. Light-source as claimed in claim 15, wherein the opening is terminated by the light-exit element in an airtight and/or watertight manner.

17. Light-source as claimed in claim 15, wherein the semiconductor element is arranged on a support arranged in the housing.

18. Light-source as claimed in claim 15, wherein the housing comprises ceramic.

19. Dental turbine handpiece or handpiece and anglepiece with an elongated gripping sleeve and a turbine head or head drive located at a front end of the gripping sleeve, the gripping sleeve comprising in its circumferential surface at least one recess in which a light-source as claimed in claim 15 is arranged.

20. Dental turbine handpiece or handpiece and anglepiece as claimed in claim 19, wherein the light-source is arranged in the vicinity of the turbine head or head drive.

21. Dental turbine handpiece as claimed in claim 19, wherein the shape of the housing of the light-source and the shape of the recess in the circumferential surface are matched to one another.

22. Light-source as claimed in claim 2, wherein the optical element comprises a light-exit window.

23. Light-source as claimed in claim 8, wherein the support is manufactured from a ceramic, silicon, or plastic electrically insulating material.

24. Light-source as claimed in claim 9, wherein the support is manufactured from a highly thermally conducting ceramic, silicon, or plastic material.

25. Light-source as claimed in claim 12, wherein the opening in the region comprises a shape that at least party corresponds to the outer shape of the support.

26. Light-source as claimed in claim 17, wherein the support is made of ceramic material.
